(19) United States
(12) Patent Application Publication
(10) Pub. No.: US 2016/0022621 A1
(43) Pub. Date: Jan. 28, 2016

(54) COMPOSITION FOR ORAL ADMINISTRATION FOR BINDING ALDEHYDES IN THE GASTROINTESTINAL TRACT

(71) Applicant: BIOHIT OYJ, Helsinki (FI)

(72) Inventor: Osmo SUOVANIEMI, Helsinki (FI)

(73) Assignee: BIOHIT OYJ, Helsinki (FI)

(21) Appl. No.: 14/774,875

(22) PCT Filed: Mar. 12, 2013
(86) PCT No.: PCT/FI2013/050274

§ 371 Date: Sep. 11, 2015

(51) Int. Cl. A61K 31/198; A61K 9/16; A61K 9/28

(52) U.S. Cl. CPC A61K 31/198; A61K 9/2826; A61K 9/2846; A61K 9/2813; A61K 9/1617

(57) ABSTRACT

The present invention relates to a non-toxic composition containing one or more cysteine compounds selected from L- or D-cysteine, N-acetyl cysteine, and the pharmaceutically acceptable salts thereof, for decreasing the risk of a subject contracting cancer of the stomach, the small intestine and the colon, by locally binding aldehydes present in the stomach, and optionally also separately the aldehydes carried to the small intestine or the colon, or both, whereby the composition is formulated with the help of two or more additives into controlled-release tablets containing at least one additive selected from cationic and gel-forming polymers, which tablets are formed from two or more separate layers with different release profiles, whereby the cysteine compounds are added both into the inner layer(s) and into the tablet material surrounding these.

### FIELD OF THE INVENTION

The present invention relates to a composition for effectively binding aldehydes in the stomach, or in both the stomach and in the small intestine or colon, of a subject (or in more than two of these areas) in order to decrease the risk of cancer in said areas of the gastrointestinal tract of said subject. The invention relates also to methods for decreasing the risk of developing cancer in the gastrointestinal tract caused by aldehydes.

### DESCRIPTION OF RELATED ART

Both alcohol and smoking are risk factors for upper digestive tract cancers, and the combined use thereof multiplies the risk of developing an upper digestive tract cancer to as much as 150-fold (Salaspuro, 2003; and Francheschi et al. 1990).

The first metabolite of ethanol, acetaldehyde, is highly toxic, mutagenic and carcinogenic, as shown in cell culture and animal experiments (IARC, 1999). Furthermore, epidemiological, genetic, microbiological and biochemical studies strongly suggest that acetaldehyde acts as a local and cumulative carcinogen in the upper digestive tract in humans (Salaspuro, 2009; Seitz and Stickel, 2010). Consequently, acetaldehyde present in alcoholic beverages and formed endogenously from ethanol was recently classified as carcinogenic to humans (group 1) by the International Agency for Research on Cancer (IARC) (Secretan et al. 2009).

Alcohol is evenly distributed in the liquid phase of the organs. Hence, after consuming alcohol, and as long as there is alcohol in the organs, the alcohol content in the blood, saliva, gastric juice and the contents of the intestine is the same. In that case, the microbes in the digestive tract are capable of oxidizing the alcohol to acetaldehyde. For example, even after a moderate dose of ethanol (0.5 g/kg), high acetaldehyde contents of a microbial origin (18-143 μM) have been found in human saliva; in other words, acetaldehyde builds up in saliva as an intermediate product of the microbial metabolism (Homann et al, 1997).

Acetaldehyde is also formed (particularly in the mouth, the pharynx, and the upper airways) as a consequence of smoking, and exposure to air contamination. It has been proven that chronic smoking significantly increases the acetaldehyde production of saliva originated in microbes. In fact, it has been demonstrated that the cancer risk associated with cigarette smoking is not only caused by the commonly known polycyclic aromatic hydrocarbons (PAH), but to a significant degree (up to 40%) by aldehydes, particularly by acetaldehyde and formaldehyde, whereas acrylic aldehyde causes up to 88.5% of the other toxic effects. Thus, a lowering of the aldehyde-content in cigarettes has been recommended (Haussmann, 2012).

The formation of acetaldehyde in the organism mainly takes place in the mouth, particularly in the saliva.

The average amount of saliva secreted by a human is 2.5 litres per day. The areas of influence of the acetaldehyde contained in the saliva include the mouth, the pharynx, the oesophagus and the stomach. Consequently, the effects of acetaldehyde may extend to the whole upper digestive tract area. On the other hand, carcinogenic acetaldehyde can be produced also endogenously by the oral microbes from various foodstuffs with high sugar or carbohydrate content, especially in an achlorhydric stomach. Atrophic gastritis and achlorhydria are well known risk factors of gastric cancer.

As a consequence of the microbial metabolism, acetaldehyde builds up in the stomach in the case, where the stomach is free from acid or has been made acid-free by medication. In the gastric juice there are for example Streptococcus viridans-bacteria, which have been shown to be excellent producers of acetaldehyde. Other effective acetaldehyde producers in acid-free stomach have been shown to be bacteria belonging to Neisseria, Rothia and Streptococcus salivarius (Väkeväinen et al., 2002).

Our recent studies show that in an achlorhydric stomach alcohol fermentation can start very quickly by the bacteria representing normal flora of the mouth or by yeasts present in the foodstuffs, for example by common baker's or brewer's yeast. These microbes can produce significant amounts of acetaldehyde and ethanol for example from carbohydrate containing foodstuffs, such as rice. This happens in particular, if the carbohydrate containing foodstuff is sweetened. For example in Asian countries the use of sweet sauces with rice is a very common practise. According to epidemiological studies the eating of rice causes a high risk for cancer in stomach.

In acid stomach the alcohol fermentation does not occur. On the other hand Helicobacter pylori infection and certain medicaments, such as Protein Pump Inhibitors (PPI) raise the pH of the stomach, whereby the same problem occurs.

One further risk factor for the stomach are foodstuffs comprising acetaldehyde. Our recent studies have shown that all sugar (saccharose, maltose, lactose) containing foodstuffs including beverages, can contain—or in the foodstuff is formed—significant amounts of acetaldehyde, 5 to 2000 μM and ethanol, 0.1 to 0.5 per mille. Some sour milks, yoghurts and juices contain acetaldehyde and ethanol as such (PCT/FI2006/000104).

It has also been shown that acetaldehyde builds up in the colon, as its bacteria that represent the normal flora are capable of converting ethanol into acetaldehyde (Jokelainen et al., 1996). In the intestines, endogenous ethanol can also be found, i.e. ethanol that is formed in the intestines in oxygen-free conditions under the effect of microbes. Acetaldehyde is formed, when this ethanol comes into contact with oxygen near the mucous membrane, for example.

The prior art discloses pharmaceutical compositions which contain compounds that bind acetaldehyde, their effect being based on the reaction of the effective substances with the acetaldehyde inside blood and/or cells, for example, U.S. Pat. No. 5,202,354, U.S. Pat. No. 4,496,548, U.S. Pat. No. 4,528,295, U.S. Pat. No. 5,922,346.

Acetaldehyde, which is formed in the organism when alcohol is consumed and thereafter, causes physiological symptoms called a hangover. Previously, efforts have been made to decrease the symptoms caused by acetaldehyde by taking preparations containing ascorbic acid, thiamine, cysteine or cysteic acid, and flavonoids or flavonoid complexes in a form of orally taken tablets in connection with, before or after consuming alcohol. When swallowed, the effective substances go to the stomach and small intestine and from there into the blood circulation (U.S. Pat. No. 5,202,354 and U.S. Pat. No. 4,496,548).

Suggestions have been made so as to use preparations containing amino acids, such as L-cysteine, methionine, taurine or arginine, ascorbic acid, vitamins A and E, which are sucked or chewed in the mouth, to reduce the effect of detrimental free radical compounds, which are formed when using tobacco products or being exposed to the same. It is believed that, after being absorbed, amino acids affect various tissues (U.S. Pat. No. 5,922,346; WO 99/00106).

WO 02/36098 suggests the use of compounds containing a free sulfhydryl and/or amino group for a local and long-term binding of acetaldehyde from saliva, the stomach or the colon. The compounds were mixed with a substance that enabled them to be released for at least 30 minutes in the conditions of the mouth, the stomach or the colon.

WO 2006/037848 suggests a composition comprising one or more free sulfhydryl and/or amino groups for removing or decreasing the aldehyde content of the saliva during smoking.

As on the basis of our recent studies, aldehydes play a considerable part in the pathogenesis of the stomach cancers, in particular by people having an achlorhydric stomach. There is thus a need to find alternative ways to bind these aldehydes in the stomach in a harmless manner. Further, there is a need for formulations that will release their contents in more than one area of the gastrointestinal tract, since also the aldehydes will be carried from the mouth further down the tract.

### BRIEF DESCRIPTION OF THE INVENTION

It is an aim of the present invention to provide new compositions, which can be used to reduce the aldehyde content in the stomach, and optionally in any of the other areas of the gastrointestinal tract. It is also an aim of the invention to provide new methods for binding aldehydes in said areas.

Particularly, it is an aim of the present invention to provide new compositions, which protect the active compound(s), for example to mask their taste or to prevent their immediate release.

These and other objects, together with the advantages thereof over known compositions and methods are achieved by the present invention, as hereinafter described and claimed.

Thus, the present invention concerns a non-toxic composition containing one or more cysteine compounds for decreasing the risk of a subject contracting cancer of the stomach, the small intestine and the colon, by locally binding aldehydes present in the stomach, and optionally also separately the aldehydes carried to the small intestine or the colon, or both.

The composition is capable of binding aldehyde present at least in the stomach, and comprises one or more aldehyde-binding compounds, which are bound at least to such non-toxic additives that effect sustained release of said active compound(s) into the stomach.

Particularly, the composition according to the invention is characterized by what is stated in the characterizing part of claim 1.

The invention provides considerable advantages. The compositions comprising aldehyde-binding compounds can be used to reduce the risk of developing cancer of the stomach, as well as of the small intestine and/or the colon of people having increased risk for cancer in these areas. By the compositions and methods of the invention can be treated in particular people suffering from atrophic gastritis, achlorhydric and low acid stomach, since these are most susceptible to said risks. In these individuals, acetaldehyde is produced locally by mouth-derived bacteria that are able to survive in the neutral environment of the stomach in that they metabolize alcohol or sugars to acetaldehyde.

Furthermore, the compositions of the present invention are effective for binding aldehyde, in particular, when they are consumed in connection with eating, or when they are consumed in connection with consuming alcohol.

The same is true for smoking or other ways of consuming or using tobacco, i.e. the compositions of the present invention are particularly effective and particularly useful for binding aldehydes when they are consumed in connection with smoking or other ways of using tobacco.

Consuming the compositions according to the invention mainly binds aldehydes locally, due to the local release in the desired areas of the gastrointestinal tract, but this also gives a systemic effect.

### BRIEF DESCRIPTION OF THE DRAWINGS

FIG. 1 shows the effect of L-cysteine administration (or placebo administration) on acetaldehyde levels.

FIG. 2 shows the mean cysteine concentrations in the gastric juice of volunteers after the administration of study formulations containing L-cysteine.

### DETAILED DESCRIPTION OF THE EMBODIMENTS OF THE INVENTION

The present invention concerns a non-toxic composition containing one or more aldehyde-binding compounds, particularly selected from cysteine compounds, for decreasing the risk of a subject (particularly a human subject) contracting cancer of the stomach, the small intestine and the colon, by locally binding aldehydes present in the stomach, and optionally also separately the aldehydes carried to the small intestine or to the colon, or both.

To achieve the local effect, the composition is formulated for controlled release of the active compound(s) with the help of two or more additives into tablets, which optionally can be coated. The tablets contain two or more separate layers with different release profiles, whereby one or more inner layers (or inner structures) preferably are in the form of granules, mini tablets, medium-sized core tablets, pellets or cross-linked matrix structures. Each layer contains at least one additive, among others for guiding the release to the desired area of the gastrointestinal tract and for providing the desired release rate. At least one of the layers contains one or more additives selected from cationic and gel-forming polymers, for providing release of the contents of said layer in the stomach.

In the following, the complete unit is called a tablet, while the inner layers are referred to as pellets or granules.

The separate layers of the tablets (the optional coating, the tablet contents, and/or the contents of the pellets or granules) are provided with different release profiles, for example by varying their solubilities (by providing an outer layer with a higher solubility in aqueous solutions compared to the inner layer(s), or by providing layers that dissolve at different pH ranges), or by varying the release rate of the separate layers (essentially by providing a quick-release outer layer and sustained-release inner layer(s), e.g. by providing the layers with different densities), whereby, in case of several inner layers, the release profiles of the inner layers can also be different.

The active compounds are used in a pharmaceutically effective amount, which for the aldehyde-binding compound(s) means an amount capable of binding or inactivating the amount of aldehyde carried to or formed in the gastrointestinal tract of a subject during the consumption of food or drinks, or during the smoking of tobacco products.

At least one of the cysteine compounds is selected from L- or D-cysteine, N-acetyl cysteine, and the pharmaceutically acceptable salts thereof, and is added both into the inner and the outer layer(s) of the tablet. Other alternative cysteine compounds can be:
cystine,
cysteic acid,
cysteine glycine,
threo or erythro-β-phenyl-DL-cysteine,
β-tetramethylene-DL-cysteine,
D-penicillamine and its dipeptides with N-terminals,
peptide or a protein with terminal cysteine,
glutathione,
reduced glutathione,
D,L-homocysteine,
D,L-homocysteic acid,
L-cysteinyl-L-valine,
β-β-tetramethylene-DL-cysteine,
cysteinyl-glycine,
tre-(5)-β-phenyl-DL-cysteine,
erythro-β-phenyl-DL-cysteine, and
cysteine hydrochloride.

Typically, a single unit, or formulation, of the composition comprises 1-500 mg, preferably 10-300 mg, more preferably 50-250 mg, and most suitably 100-200 mg of the cysteine compound(s). However, 1-2 of these units can be administered at once.

The total content of the cysteine compound(s) is then 1 to 40 w-%, preferably 5 to 40, more preferably 10 to 30 w-%. Typically the amount is 20 to 25 w-%.

The composition of the invention comprises at least one non-toxic additive that causes sustained release of the aldehyde-binding compound(s) in the stomach. Sustained release here means the release of said aldehyde-binding compound(s) for at least 30 minutes in the conditions of the stomach. Preferably the compound(s) are released during 0.5 to 8 hours, preferably 2 to 6 hours, most preferably 2 to 4 hours. The optional tablet coating and possibly the size of the tablet already cause controlled delay in the release. However, it is preferred to use also other additives delaying the release of the active compound(s).

The term “additive” here includes carriers, fillers, binders and coatings, as well as aromatic agents, colorants and non-functional additives. These additives are non-toxic, and preferably at least a portion of them function by controlling the release of the active compound(s) to take place specifically in the stomach, or in the small intestine or the colon, and most suitably in a sustained manner. Another objective of using said additive(s) is to protect the active compound(s), among others to mask their taste.

Preferably, the additives are selected to achieve the release of pharmaceutically active cysteine compound(s) in the conditions of the stomach in an amount of 40-80 mg in an hour.

For example, two L-cysteine molecules readily react with eachother to form cystine (by dimerizing) that is not able to effectively bind and inactivate the aldehyde present in the stomach (or the other areas of the gastrointestinal tract), particularly when used alone in a conventional immediate-release dosage form. However, the present invention provides a dosage form (particularly a large coated tablet to be swallowed by the subject) that gives a long-term effect, and prevents said dimerization. Furthermore, acetaldehyde is produced during the entire time that the food stuff and/or alcohol resides in the stomach. Thus, the said formulation provides sustained source of L-cysteine over the expected time of acetaldehyde exposure.

Preferably, the used additives are selected from those capable of controlling the release of the active compound(s) and the granules so that these are released locally in the stomach during a time of more than 30 minutes, preferably 0.5-8 hours, most suitably in 2-4 hours.

According to a preferred embodiment of the present invention, the composition is administered in connection with eating, i.e. just before, during or just after eating, or in connection with consuming alcohol, i.e. just before, during or after consuming a dose of alcohol.

According to another preferred embodiment of the invention, the composition is administered in connection with smoking or other use of tobacco, i.e. just before, during or just after smoking (or other use of tobacco).

A further advantageous option is to administer one dose (e.g. one unit) of the composition just before and another dose just after eating, drinking or smoking.

The terms “just before” and “just after” mean a time frame of up to 5 minutes before or after eating, consuming alcohol or smoking (or otherwise using tobacco), preferably a time frame of up to 2 minutes, more preferably a time frame of up to 1 minute, and most suitably a time frame of up to 0.5 minutes before or after eating, consuming alcohol or smoking.

However, the compositions can also be used in a continuous way, for example every 10 minutes. According to a preferred embodiment of the invention the dosage is renewed at 5- to 15-minute intervals, preferably at 5- to 10-minute intervals, if alcohol consumption or smoking is continued for an interval longer than the said one. Alternatively, the composition can be administered at 4- to 10-hour intervals, preferably at 6- to 8-hour intervals.

“Smoking” refers to the smoking of any tobacco product, using snuff, chewing tobacco, or any other use of a tobacco product, wherein the tobacco product or a part thereof is placed in the mouth or in close vicinity to the oral cavities. The tobacco product can thus be a cigarette, a cigar, snuff, chewing tobacco or pipe tobacco.

The “binding of aldehyde” preferably refers to a chemical reaction between the aldehyde and the free sulphhydryl or amino group, or both, of the cysteine (or similar compound), wherein the aldehyde jointly with the “aldehyde-binding compound” forms a larger molecule. In the reaction with cysteine, for example acetaldehyde mainly binds itself to the sulphhydryl and the amino group of the cysteine, and forms 2-methyl-L-thiazolidine-4-carboxylic acid (and water).

According to the invention, the compounds obtained from aldehydes by chemical binding with cysteine, or a derivative or salt thereof, are safe for the subject.

However, the aldehydes (in free form) are not safe for the subject. A harmful/carcinogenic content of acetaldehyde in e.g. the human mouth is roughly 20 to 800 μmol/l of saliva, and a content of as low as about 20 to 50 μM causes carcinogenic DNA adducts on the cell level. Generally, levels of above 40 to 100 μM are considered mutagenic. Further, formaldehyde is responsible for some carcinogenic effect, while acrolein causes other toxic effects. Since the saliva that is carried to the stomach is also the main cause of the aldehyde-contents of the stomach, similar values are valid also for the present invention.

By administering the composition of the invention, the aldehyde content in the stomach can be reduced to a level that is essentially lower than without the use of the composition, which means that the aldehyde content can be kept at a level that is at least 20% lower, preferably >40% lower, and most suitably >60% lower than in a corresponding situation without using the composition according to the invention.

Such a harmful or carcinogenic content of aldehyde in the human stomach, as well as in the other parts of the gastrointestinal tract, can be caused by consuming alcoholic drinks, particularly strong alcoholic drinks, or foodstuffs containing alcohol, or as a consequence of smoking, or when consuming products (e.g. foodstuffs) containing aldehyde.

The aldehydes can be formed from the ethanol generated by oral microbes. Both these microbes and the formed aldehydes are constantly carried to the stomach with the saliva, particularly as the subject swallows. The “oral microbes” are intended to include oral bacteria and microbes in the oral cavity, such as streptococci, lactobacilli, corynebacteria, oral spirochetes, anaerobic cocci, and specifically Porphyromonas gingivalis, and various Candida species, including C. glabrata, C. parapsilosis, C. tropicalis, C. dubliniensis, C. guilliermondii, C. albicans, and C. krusei (in the pharynx). However, the aldehyde can also be contained in the alcoholic drink or in the foodstuff, either as product of the manufacturing process, i.e. being a fermentation product, or it can be added as such into the drink or the foodstuff. Further, some of the generated aldehydes are carried further, as far as the small intestine, or even the colon, particularly if they have not been bound or inactivated in the stomach.

“Alcoholic drinks” are ethanol-containing drinks, their ethanol content varying within 0.7% by volume and 84% by volume.”

“Alcoholic foodstuffs” refer to foodstuffs containing at least 0.7% by weight of ethanol. Such foodstuffs can be, for example, fermented juices, yoghurts, any pickled food or other kind of preserves, or foodstuffs preserved with small amounts of alcohol, pastries, jellies, and mousse seasoned with liqueur or corresponding products containing alcohol.

“Aldehyde-containing foodstuffs” refers to foodstuffs containing aldehyde even prior to consumption (i.e. in contrast to the aldehyde formed in the mouth of a subject consuming said foodstuff). Among others, acetaldehyde can be formed in said foodstuffs from ethanol that is generated in connection with fermentation, such as in beer, cider, wine, home-brewed beer, and other alcoholic drinks, as well as in many juices. In certain foodstuffs, such as some dairy products, this acetaldehyde is used for preservation purposes and to add flavour, or the acetaldehyde is formed in the product as a consequence of microbial activity. For example, sugary juices or sugar-containing foodstuffs, in general, provide a suitable substrate for acetaldehyde-producing microbes. High concentrations of acetaldehyde are also formed in fermented dairy products, such as yoghurt. In that case, mainly the microbes used to make yoghurt produce the acetaldehyde. As for alcoholic drinks, sherry and Calvados contain also especially large amounts of acetaldehyde.

The use of the compositions according to the invention can be of benefit even, when light alcoholic drinks or foodstuffs are consumed, i.e. those containing only small amounts of alcohol (even <0.7%), since even these contents are carcinogenic in the long run.

The additives in the composition include a combination of substances, which can function specifically as carriers, fillers, binders, coatings, aromatic agents, and other types of additives.

According to an alternative of the invention, the composition is formulated into a coated tablet, with at least one of the additives forming said coating (e.g. a polymeric or inorganic film coating or sugar coating). Preferably, such a tablet is compressed from the constituents of the composition (excluding the coating additive), which constituents can include the above described inner layer(s), whereby each inner layer can optionally be covered with a further polymeric film.

In case of the inner layer(s) being in the form of granules, they can be prepared by moisturing a dry mixture of the desired granule contents, and granulating it by using methods and devices that are well-known in the pharmaceutical industry. Inner tablets or pellets can be prepared by compressing, also using methods and devices that are well-known in the pharmaceutical industry.

The tablets and the pellets can be shaped as discs or as capsules, or they can be round or oval shaped.

It is of advantage if the composition is formulated into a large tablet, the diameter of which is at least 7 mm, preferably 8 to 15 mm, more preferably 11 to 15 mm. For an oval or capsule shaped tablet, the length can, in turn, be as high as 20 mm. The large diameter delays the dissolution of the tablet, and the escape from the stomach.

However, the composition can also be formulated into a small tablet or a lozenge-like structure, having a diameter of <5 mm, preferably 2 to 4 mm, most suitably 2 to 3 mm. Such a formulation is particularly suitable for use with small amounts of active compound(s), such as <100 mg of cysteine compound(s), preferably ≦80 mg, and most suitably ≦50 mg. Such small amounts are sufficient at least for binding aldehydes in the mouth, whereby at least an outer layer of the tablet could be formulated for release in the mouth, whereas the contents including the one or more inner layers could be swallowed by the subject, after dissolution of the outer layer, to provide the desired effect further down the gastrointestinal tract. However, due to the small size of such tablets, they can easily be administered in amounts of >1 tablet per dose, whereby a sufficient amount of active compound(s) can be provided also for the other areas of the gastrointestinal tract.

Said small tablets have the advantage of being easy to swallow, and can even be mixed with food to provide a particularly suitable time of administration.

According to an embodiment of the invention, the composition comprises at least one additive that causes the quick (possibly even immediate) release of a first portion of the active compound(s) and at least one additive that delays the release of a second portion of the active compound(s). Thus, it is preferred to formulate at least one inner layer to provide the delayed (i.e. sustained) release and an outer layer to provide the quick release. The quick release can be provided, for example with any common water-soluble tablet coating (including coatings made of carbohydrates, inorganic salts and water-soluble polymers). Such a formulation is particularly suitable for release of the active compound(s) in the stomach, thus providing a longer period of action of the active compound(s).

According to a preferred embodiment of the invention, the composition comprises at least one additive that does not dissolve or dissolves only poorly in the stomach. One option for achieving this is to cover the composition (the tablet or an inner layer thereof) with a film-layer that is scarcely soluble in water.

According to another preferred embodiment of the invention, the composition comprises at least one additive selected from those that form a gel in the conditions of the stomach, which gel then helps to maintain the formulation floating in the contents of the stomach for a prolonged time. Such a gel-forming additive can be added into a dry mixture to be granulated, or into a mixture to be compressed.

Suitable additives not dissolving in the stomach include polymers, such as a hydroxypropylmethyl cellulose, polypropylene, Carbopol, or metacrylate polymer, for example Eudragit RS or S, or ethyl cellulose. Additives for use in achieving a gel are various chitosans, alginates, such as sodium alginate, aluminium hydroxide, sodium hydrogen carbonate, sodium carboxymethyl cellulose, and sodium hydrogen carbonate as described in WO 02/36098.

It is particularly preferred to achieve a formulation that floats in the contents of the stomach by using polymers, such as alginic acid, as additives, which form a gel, and by adding to the formulation sodium hydrogen carbonate, which under the influence of gastric acid releases carbon dioxide, which in turn forms gas bubbles inside the gel.

A gel that floats in the stomach can also be prepared from sodium alginate, aluminium hydroxide, sodium hydrogen carbonate, and water, to which the aldehyde-binding compound(s), or the granules formed thereof, can be added.

A corresponding formulation is also obtained by adding the aldehyde-binding compound(s), or the granules formed thereof, to an aqueous dispersion of chitosan.

The content of said polymers, or other gel-forming agents, in the composition is preferably 10 to 50 w-%, more preferably 20 to 40 w-%, and most suitably 20 to 30 w-%.

According to a second preferred embodiment, the composition contains one or more additives, e.g. in the form of a coating (preferably on an inner layer of the tablet), having the task of carrying a portion of the composition (including a portion of the active compound(s)) past the stomach. Such additives are preferably selected from enteric substances, such as enteric polymers, which preferably are selected from methacrylate derivatives, or hydroxypropyl methylcellulose phthalate, hydroxypropyl methylcellulose succinate or hydroxypropyl methylcellulose acetate-succinate. These are resistant to stomach acid, and dissolve in the less acidic area of the intestines.

The above described enteric polymers can be divided into two groups, whereby Eudragit RL, RS, and NE are used for controlled release or sustained release formulations (or selected tablet layers), and function e.g. based on their insolubility and by swelling, whereas Eudragit L and S are used for colon or intestine targeted formulations (or selected tablet layers), since they are pH dependent. Eudragit L is particularly suitable for targeting the release of active compound(s) in the small intestine, while Eudragit S is particularly suitable for targeting the release of the active compound(s) in the colon. The function of these pH dependent additives is based on their ionizable carboxylic groups that become ionized at specific pH values, thereby making said polymers soluble. Eudragit L thus dissolves at a pH value above 5.5, while Eudragit S dissolves at a pH value above 7.

The additives of the composition can also include one or more inert bulking agents or fillers, preferably selected from calcium hydrogen phosphate, microcrystalline cellulose (MCC), lactose, or other corresponding bulking agents that are either water-soluble or non-soluble in water. Particularly, the bulking agents are selected from non-swelling agents.

The content of such optional bulking agents in the composition is preferably 20-70 w-%, more preferably 40 to 60 w-%, and most suitably about 50 w-%.

Further, the additives of the composition can include one or more aromatic agents, such as flavourings, particularly as a coating on the outermost layer of the tablet. Such an aromatizing coating is particularly suitable for use on tablets intended to release a portion of their contents in the mouth. Typical aromatic agents include carbohydrates (or sugars), such as glucose, sorbitol, eucalyptol, thymol, sucrose, sodium saccharine, methyl salicylate, menthol and xylitol, and are preferably selected from glucose, sorbitol, sucrose and xylitol.

Using the above described embodiments and the layered tablet structure, the composition of the present invention can be formulated to provide a tablet releasing portions of the active compound(s) in 2 to 4 distinct areas of the gastrointestinal tract. Any combinations can be provided, but preferred combinations include an effect in:
the mouth and the stomach,
the stomach and the small intestine
the stomach and the colon,
the stomach, as well as the small intestine and the colon, and
the mouth, the stomach, and the small intestine and the colon.

All of the above combinations are preferably achieved using additives that provide sustained release in said areas of the gastrointestinal tract.

As stated above, it is an option to formulate the composition by granulating a portion of its components, and pressing these (possibly together with a further portion of the components) into tablets that preferably are then coated. The granulation can be carried out by using enteric polymers as binders. These are preferably selected from polymers with a solution pH of 6-7, and most preferably from methacrylate derivatives, which are known by the trade names Eudragit L, Eudragit S, and Eudragit RS. The amount of enteric polymer in the preparation is preferably 2-5%, most preferably 3-4%.

However, according to another option, the composition of the invention is formulated into a tablet containing one or more separate small core tablets or pellets.

Any such tablets are preferably coated with a polymeric film. Similarly, the granules can optionally be coated with a further polymeric film. Such polymeric films can be formed using porous film forming agents, such as ethyl cellulose (EC) and hydroxypropyl methylcellulose (HPMC). Preferably a mixture of these are used, more preferably a mixture, where the relative amount of EC to HPMC is 1/1 to 5/1, preferably 2/1 to 5/1, and most suitably 3/2 to 7/3. Such a mixture has advantageous dissolution properties due to the different characters of the constituents, as HPMC is a water-soluble polymer and EC is a water-insoluble polymer. In the conditions of the stomach the water-soluble polymer dissolves and pores are formed to the water insoluble polymer. In such a case, the release of the highly water-soluble cysteine compound(s) is based on its diffusion from the pores formed to the film. The film-forming substances also effectively mask the taste of the cysteine compound(s).

A typical composition can comprise for example:

| | |
|---|---|
| Aldehyde binding compound(s) | 5 to 40 w-% (preferably 25 w-%) |
| Polymer not dissolving in | 10 to 50 w-% (preferably 20 to 30 w-%) |
| stomach | |
| Inert bulking agent | 20 to 70 w-% (preferably 40 to 60 w-%) |
| Ethanol | q.s. |

In such a formulation, the release of the active compound(s) is mainly based on the diffusion of the water-soluble active compound(s) from the pores formed to the tablet matrix. In case the tablet is coated, this can be carried out separately, preferably using a water-soluble polymer, such as hydroxypropyl methyl cellulose (HPMC), and a water-insoluble polymer, such as ethyl cellulose (EC). In the conditions of the stomach the water-soluble polymer dissolves and pores are formed to the water insoluble polymer.

According to a particularly preferred embodiment, the composition is formulated to release a portion of the active compound(s) in the small intestine. This requires said portion of the active compound(s) to be protected from release in the stomach.

Said protection of the active compound(s) can be applied, for example, using a polymer, such as Eudragit L, that dissolves in an environment with a pH above 5. Such a polymer is preferably applied as a film on an inner structure (e.g. granules, core tablets or pellets) inside the above described main tablet, whereby the substances surrounding the inner structure are released in the stomach, due to the selection of the contents and coatings on the main tablet, while the substances inside the inner structure are released in the small intestine, due to the selection of their separate film coating.

Said composition is prepared either by mixing the granules or pre-formed core tablets or pellets (containing a portion of the active compounds) with the remaining portion of the active compound(s) as well as with optional additives (selected to achieve release of the contents in the stomach), and the formed mixture is compressed into main tablets, which are optionally coated.

The ratio of the cysteine compound(s) intended for release in stomach and in the small intestine is preferably 1:1 to 1:3, typically 1:2, due to, among others, the differences in the volume of these areas of the GI tract.

According to another particularly preferred embodiment, the composition is formulated to release a portion of the active compound(s) in the colon. This requires said portion of the active compound(s) to be protected both from release in the stomach and from release in the small intestine.

Said protection of the active compound(s) can be applied, for example, using a polymer that dissolves in an environment with a pH of above 6.5, preferably 6.5-7.0, such as Eudragit S. Such a polymer is preferably applied as a film coating, which does not dissolve in the acidic environment of the stomach, but dissolves at a pH value of 7.5, at the latest, is preferably applied on an inner structure (such as granules, core tablets or pellets), whereby the substances surrounding the inner structure are released in the stomach, due to the selection of the contents and coatings on the main tablets, while the substances inside the inner structure are released in the colon, due to the selection of their separate film coating.

It is also possible to replace the above polymer film coating with polysaccharides that degrade under the effect of microbes of colon, or polymers generated by azo bonds.

Further useful enteric polymers include the various grades of hydroxypropyl methylcellulose phthalate, hydroxypropyl methylcellulose succinate or hydroxypropyl methylcellulose acetate-succinate (HPMC-AS) or the like, such as sold by the trade name Agoat™, Aqoat AS-HF™ in particular, a cellulose acetatephtalate (CAP) grade sold by the trade name Aquateric™, and methacrylic acid derivative, methacrylic acid methyl methacrylate copolymers. In addition to the above mentioned preferred polymers, any mixtures of these can be used.

Thus, according to said particularly preferred embodiment of the invention, the composition comprises a tablet, as described above, containing an inner structure including enteric polymers that are carried past the stomach. The enteric inner structure can comprise for example:

| | |
|---|---|
| Aldehyde-binding substance | 100 mg |
| Filler, e.g., calcium hydrogen phosphate | 30-50 mg |
| Enteric polymers | 40-60 mg |

These inner structures are mixed with the remaining portion of the active compound(s) and optional additives (selected to achieve release of the contents in the stomach), and the formed mixture is compressed into tablets, which are optionally coated.

The ratio of the cysteine compound(s) intended for release in stomach and in the colon is preferably 1:1 to 1:3, typically 1:2, due to, among others, the differences in the volume of these areas of the GI tract.

According to a further particularly preferred embodiment, the composition is formulated to release one portion of the active compound(s) in the stomach, one portion in the colon and one portion in the small intestine. Typically, such a formulation is prepared by forming two types of inner structures, according to the above preferred embodiments, one for release in the colon and one for release in the small intestine, mixing these inner structures with the third portion of active compound(s) and optional additives (selected to achieve release of the contents in the stomach), which mixed contents are all compressed into main tablets (that are optionally coated).

The ratio of the cysteine compound(s) intended for release in stomach and of the cysteine compound(s) intended for release in the small intestine and the colon is preferably 2:1:1 to 1:3:3, typically 2:1:1, 1:1:1, 1:1:2, 1:1:3, 1:2:2, 1:2:3, 1:1:3 or 1:3:3, particularly 1:2:2 or 1:3:3, and most suitably 1:2:2.

In addition to the above active compounds and additives, it is of advantage to add to the compositions of the present invention at least one of the substances selected from the group comprising xylitol, sulphites, chromium, vitamin B12, A-, D-, E, -C-vitamins, niacin, biotin, thiamine, B2-, B5-, B6-vitamins and folic acid and trace elements, such as chromium, manganese, selenium, zink and iron.

Further, according to one preferred embodiment of the invention, the composition is administered to people having an achlorhydric stomach in connection with administering medication that causes said achlorhydric stomach, such as proton pump inhibitors. One component of gastric juice is hydrochloric acid (HCl), the secretory product of the parietal, or oxyntic cell of the corpus of the stomach. It is known that the capacity of the stomach to secrete HCl is almost linearly related to parietal cell numbers (Yao et al. 2003, Samuelson et al. 2003). Acid secretion is dependent on function of the H+/K+ATPase or proton pump located in the cannilicular membrane of the parietal cell.

Several drugs have been developed that non-competitively bind and inactivate the ATPase, resulting in strong inhibition of acid secretion. Such drugs include proton pump inhibitors (PPIs), such as:
Dexlansoprazol
Esomeprazol
Lansoprazol
Omeprazol
Pantoprazol
Rabeprazol
Tenatoprazol

For example, omeprazole (such as Prilosec) is an acid-activated prodrug that binds covalently to two cysteines on the ATPase, resulting in its irreversible inactivation. Other proton pump inhibitors (PPIs), including lansoprazole (Prevacid), esomeprazole (Nexium), rabeprazole (Aciphex) and pantoprazole (Protonix) have similar modes of action (Hellstrom et al. 2004, Sachs et al. 1994, Shamburek et al. 1992, Welag et al. 2003).

According to a particularly preferred embodiment of the present invention, the composition includes (in addition to the aldehyde-binding compounds), one or more of said PPIs, whereby a combination product is obtained that is intended for ameliorating the symptoms of a hyperchlorohydric stomach, an H. pylori infection, gastroesophageal or oesophageal reflux disease or atrophic gastritis.

The following non-limiting examples demonstrate the advantages obtained with the preferred embodiments of the invention.

### EXAMPLES

### Example 1

### Acetaldehyde-Binding

An analysis was carried out after administering L-cysteine (in a total of 200 mg) or placebo, orally to subjects. This cysteine or placebo was formulated into film-coated tablets as described above, with the cysteine or the placebo in granules together with a polymer not dissolving in the stomach and an inert bulking agent. The subjects swallowed said tablets with 200 ml of water. Immediately thereafter they were given ethanol (0.3 g/kg body weight) diluted in water to 15 vol %.

Gastric juice samples were collected, and their L-cysteine concentrations were determined using HPLC, and two parallel samples.

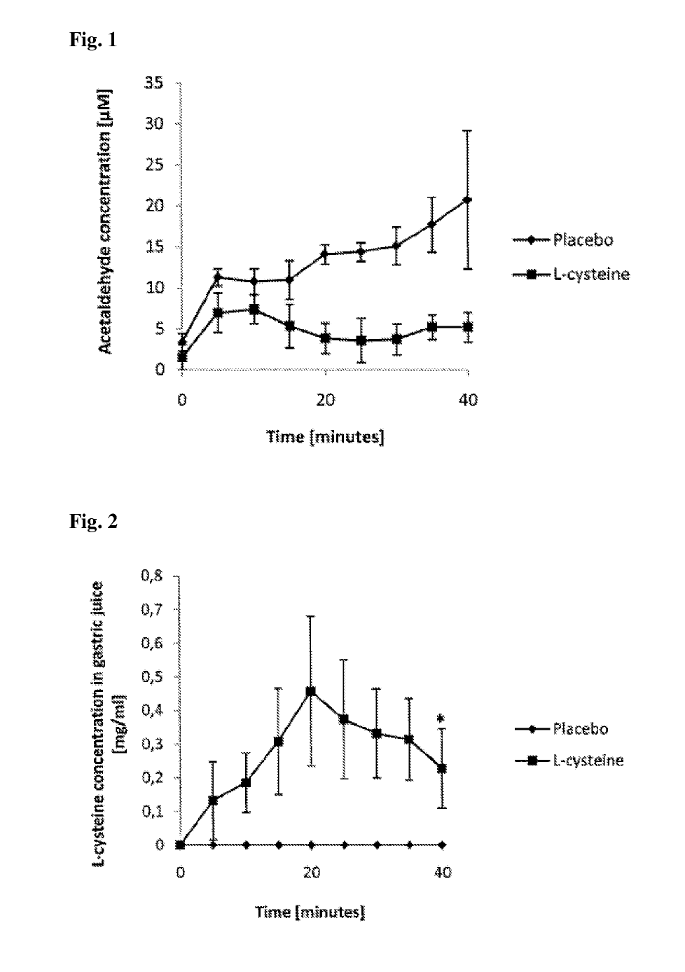

FIG. 1 shows the effect of the L-cysteine administration (or the placebo administration) on the acetaldehyde levels in the stomach of the subjects. In all measurements, the average acetaldehyde concentration of the gastric juice was 2.6 times higher with placebo than with cysteine. No significant differences existed in ethanol concentrations between cysteine and placebo treatments. The average ethanol concentration in the gastric juice was 5.0 vol-% in the first sample, declining to 0.9 vol-% in the 40-minute sample. A positive correlation emerged between the acetaldehyde concentration and the ethanol concentration.

L-cysteine was detected in the gastric juice of all subjects after the administration of study formulations containing L-cysteine. The mean cysteine concentrations are represented in FIG. 2. After administration of placebo formulations, no L-cysteine was detected. No significant correlation was found between the cysteine concentration and the acetaldehyde concentration.

### LITERATURE

Francheschi et al. Cancer Res 1990; 50:6502-07
Haussmann, H.-J. Chem. Res. Toxicol. 2012; 25, 794-810
Hellstrom P M, Vitols S: The choice of proton pump inhibitor: does it matter? Basic Clin Pharmacol Toxicol 2004; 94:105
Homann et al, Carcinogenesis 1997; 18; 1739-1743
International Agency for Research on Cancer, 1999; Acetaldehyde. In: Re-evaluation of some organic chemicals, hydrazine and hydrogen peroxide. IARC Monographs on the evaluation on the Carcinogenic Risks to Humans, vol 71, pp. 319-335.
Jokelainen et al. Gut 1996; 39:100-104
Sachs G, Prinz C, Loo D, etc: Gastric acid secretion: activation and inhibition. Yale J Biol Med 1994; 67:81-95
Salaspuro, M. Best Pract Res Clin. Gastroenterol 2003; 17:679-94
Salaspuro M. Acetaldehyde as a common denominator and cumulative carcinogen in digestive tract cancers. Scand J Gastroenterol 2009; 44: 912-925.
Samuelson L C, Hinkle K L: Insights into the regulation of gastric acid secretion through analysis of genetically engineered mice. Annu Rev Physiol 2003; 65:383-400
Secretan B, Straif K, Baan R, Grosse Y, El Ghissassi, Bouvard V et al. A review of human carcinogens—Part E: tobacco, areca nut, alcohol, coal smoke, and salted fish. Lancet Oncol 2009; 10: 1033-1034.
Seitz H K, Stickel F. Acetaldehyde as an underestimated risk factor for cancer development: role of genetics in ethanol metabolism. Genes Nutr 2010; 5: 121-128.
Shamburek R D, Schubert M L: Control of gastric acid secretion. Histamine H2-receptor antagonists and H+K(+)-ATPase inhibitors. Gastroenterology Clinics of North America 1992; 21:527-550
Väkeväinen et al. Scand. J. Gastroenterol 2002; 37:648-655
Welage L S: Pharmacologic properties of proton pump inhibitors. Pharmacotherapy 2003; 23:74S-80S
Yao X, Forte J G: Cell biology of acid secretion by the parietal cell. Annu Rev Physiol 2003; 65:103-131

1. A non-toxic composition containing one or more cysteine compounds for decreasing the risk of a subject contracting cancer of the stomach, the small intestine and the colon, by locally binding aldehydes present in the stomach, and optionally also separately the aldehydes carried to the small intestine or the colon, or both,
characterized in that
the composition is formulated with the help of two or more additives into controlled-release tablets containing at least one additive selected from cationic and gel-forming polymers, which tablets are formed from two or more separate layers with different release profiles, and
the cysteine compound(s) are selected from L- or D-cysteine, N-acetyl cysteine, and the pharmaceutically acceptable salts thereof, and are added both into the inner structure(s) and into the tablet material surrounding these.

2. The composition according to claim 1, having a content of cysteine compound(s) of 1 to 40 w-%, preferably 5 to 40 w-%, more preferably 10 to 30 w-%, and typically 20 to 25 w-%.

3. The composition according to claim 1, which comprises 1-500 mg, preferably 10-300 mg, most suitably 100-200 mg of cysteine compound(s) per single dose.

4. The composition according to claim 1, wherein the separate layers include one or more inner layers or inner structures in the form of granules, mini tablets, medium-sized core tablets, pellets or cross-linked matrix structures.

5. The composition according to claim 1, wherein each layer contains at least one additive, among others for guiding the release to the desired area of the gastrointestinal tract and for providing the desired release rate.

6. The composition according to claim 1, wherein the cationic and gel-forming polymers are selected from matrix-forming polymers, such as methacrylate polymers, ethyl cellulose, polypropylene, Carbopol, hydroxy-propylmethyl cellulose, sodium carboxymethyl cellulose, chitosans, and alginates, preferably from the methacrylate derivatives Eudragit L, S, RL, RS or NE.

7. The composition according to claim 6, which further includes one or more non-polymeric gel-forming additives selected from aluminium hydroxide and sodium hydrogen carbonate.

8. The composition according to claim 1, wherein the amount of cationic and gel-forming polymer(s) and optional non-polymeric gel-forming additives is 10-50 w-%, preferably 20-40 w-%, and most suitably 20-30 w-%.

9. The composition according to claim 1, which is in the form of a tablet having a diameter of ≧7 mm, preferably 8 to 15 mm, and most suitably 11 to 15 mm.

10. The composition according to claim 1, which is in the form of a small tablet or a lozenge-like structure, having a diameter of <5 mm, preferably 2 to 4 mm, most suitably 2 to 3 mm.

11. The composition according to claim 1, wherein the additives of the tablet are selected so that its contents are released in the stomach for a time period of at least 30 minutes, preferably for 0.5 to 8 hours, most suitably for 2 to 6 hours.

12. The composition according to claim 1, which is formulated into a tablet, with at least one additive being in the form of a film coating, preferably a polymeric or inorganic film coating or a sugar coating, more preferably a water-soluble film made of hydroxypropyl methylcellulose (HPMC) or gelatin or a mixture of these, or a water-insoluble film made of ethyl cellulose or Eudragit RS or a mixture of these, or with both said films.

13. The composition according to claim 1, which contains one or more additives, particularly on one or more inner layers of the tablet, for carrying a portion of the composition past the stomach, to the small intestine, the colon or both, these additives preferably being selected from enteric substances, such as enteric polymers, which more preferably are selected from methacrylate derivatives, or hydroxypropyl methylcellulose phthalate, hydroxypropyl methylcellulose succinate or hydroxypropyl methylcellulose acetate-succinate.

14. The composition according to claim 13, wherein the enteric polymers are selected from polymers with a solution pH of 5-8, preferably about 6 or about 7, most suitably from Eudragit L and Eudragit S.

15. The composition according to claim 13, wherein the content of enteric polymer(s) is 2-5%, preferably 3-4%, calculated from the weight of the composition.

16. The composition according to claim 1, wherein one or more inner layers is formulated to include a water-soluble polymer, such as hydroxypropyl methylcellulose (HPMC) or gelatin, preferably in the form of a film coating.

17. The composition according to claim 1, wherein one or more inner layers is formulated to include a polymer that dissolves in an environment having a pH value above 6.5, preferably selected from Eudragit S, or a combination of said methacrylate derivative with another enteric polymer, most suitably in the form of a film coating.

18. The composition according to claim 1, wherein one or more inner layers is formulated to include a polymer that dissolves in an environment having a pH value of above 5.5, preferably selected from Eudragit L or a combination of said methacrylate derivative with another enteric polymer, most suitably in the form of a film coating.

19. The composition according to claim 1, wherein the relative amount of active compound(s) for the tablet and for the inner layer(s) is from 1:1 to 1:3, preferably 1:2.

20. The composition according to claim 1, which contains two types of inner layers, the first type formulated to include a polymer that dissolves in an environment having a pH value above 6.5, and the second type formulated to include a polymer that dissolves in an environment having a pH value above 5.5.

21. The composition according to claim 20, wherein the relative amounts of active compound(s) for the tablet and for the inner layers of the first type and of the second type is 2:1:1, 1:1:1, 1:1:2, 1:2:2, 1:2:3 or 1:1:3, preferably 1:1:1 or 1:2:2, most suitably 1:2:2.

22. A method for decreasing the risk of a subject contracting cancer of the stomach, the small intestine and the colon, by locally binding aldehydes present in the stomach, and optionally also separately the aldehydes carried to the small intestine or the colon, or both, wherein a non-toxic composition is administered to a subject, which composition has been formulated with the help of two or more additives into controlled-release tablets containing at least one additive selected from cationic and gel-forming polymers, which tablets are formed from two or more separate layers with different release profiles, and wherein the cysteine compounds are selected from L- or D-cysteine, N-acetyl cysteine, and the pharmaceutically acceptable salts thereof, and are added both into the inner structure(s) and into the tablet material surrounding these.
